(19) United States
(12) Patent Application Publication
(10) Pub. No.: US 2011/0165243 A1
(43) Pub. Date: Jul. 7, 2011

(54) Concurrent delivery of multiple therapeutic agents via hydrogels for biomedical applications

(76) Inventor: Elizabeth Barker, Southaven, MS (US)

(21) Appl. No.: 12/928,206

(22) Filed: Dec. 7, 2010

Related U.S. Application Data
(60) Provisional application No. 61/283,864, filed on Jan. 4, 2010.
(60) Provisional application No. 61/397,001, filed on Jun. 7, 2010.

(51) Int. Cl. A61K 9/00; A61K 35/12; A61K 35/30; A61P 35/00; A61P 25/28

(52) U.S. Cl. 424/484; 424/93.7

(57) ABSTRACT

A process for concurrent delivery of multiple therapeutic agents via hydrogels. The process includes polymerizing a hydrogel and combining said hydrogel with at least two biologically therapeutic agents. Encapsulation of whole viable cells into polymerized hydrogel for transplantation is also disclosed.

This is a Provisional Patent Application filed for the invention by Elizabeth Barker, of 6515 Evergreen Street, Southaven, Miss. 38671, for a new and useful “Concurrent delivery of multiple therapeutic agents via hydrogels for biomedical applications.”

### CLAIM OF PRIORITY

The applicant claims for this application the priority date established by provisional patent applications 61/283,864, filed on Dec. 10, 2009, and 61/397,001, filed on Jun. 7, 2010.

### FIELD OF THE INVENTION

The invention relates to the use of biodegradable carbohydrate-based hydrogels as delivery systems for concurrent delivery of more than one therapeutic agent for biomedical applications including, but not limited to delivery systems for whole, viable biological cells.

### BACKGROUND OF THE INVENTION

Modern medicine now employs combination therapy regimens to manage many disease states including Diabetes, Heart Disease, and Cancer. Multiple therapeutic agents are often employed to treat many symptoms as well as causes of disease, however drug properties and patient compliance of several medications impedes the success of many of these regimens. There is certainly a need for novel materials that allow for simultaneous release of multiple therapeutic compounds from a single drug delivery system to improve both patient compliance and therapeutic efficacy.

For example, low drug penetration and drug resistance complicates treatment of solid tumors. These problems are increased by the presence of multiple cell types in solid tumors. The presence of multiple cell types creates a need for delivery of more than one therapeutic compound via single delivery mechanism. This concurrent, combinatorial therapy has been shown to increase efficacy of chemotherapy. (Bae et al, J. Cont. Rls., 122 (2007) 324-330; Hirsch et al, Cancer Res., 69 (2009); (19) 7507-11; Bouhadir et al, Biomaterials 22 (2001); 2625-2633). Not only is simultaneous release important for eradication of tumors, but also the advantage of a gel system is that the hydrogel can be injected locally at a solid tumor site to increase chemotherapy concentration at the target tissue and decrease drug concentrations at non-target tissues preventing drug toxicity to healthy tissues as well as decreasing side effects.

Concurrent delivery of multiple therapeutic compounds, with different mechanisms of action, has been found to create synergistic effects in treating cancer and preventing metastasis. Id. This synergistic effect not only leads to more efficacious treatment, but allows for lower-dose treatments, thereby lowering toxicity to non-cancerous tissues.

In order to more effectively treat multiple cell types, take advantage of synergistic drug interactions, and allow for lower dose treatments, methods of concurrent delivery of multiple therapeutic agents is desirable.

Recently whole living cells have been increasingly exploited as alternative drug delivery devices. Cells can act as drug depots enabling the delivery of therapeutic molecules over an extended time period. Cells are capable of delivering drugs in response to an external stimulus, which is highly advantageous to maintain homeostasis for patients suffering from chronic diseases, like diabetes or Parkinson's disease. In addition, cells can be made to secrete therapeutic proteins, cytokines, and other medically useful substances that cannot be artificially synthesized outside a living cell. Therefore, cell transplantation as a means of delivering such useful substances provides a mechanism to treat various conditions and pathologies that cannot be treated through other technologies. A variety of stem cells, progenitor cells, lineage committed cells, and genetically engineered cells have been tested in preclinical and clinical trials as drug delivery vehicles. Stem and progenitor cells secrete a diverse array of growth factors, including vascular endothelial growth factor (VEGF) and nerve growth factor, which are used to treat ischemia and neuronal damage, respectively. Islet cells, kidney cells, and parathyroid cells are examples of lineage-committed cells, which naturally secrete insulin, erythropoietin, and parathyroid hormone, respectively. These therapeutic proteins are used in the treatment of diabetes, anemia, and hyperthyroidism. Many cells, such as kidney cells, ovary cells, fibroblasts, and myoblasts, have been genetically engineered to secrete specific therapeutic proteins. Such proteins include endostatin to suppress cancer metastasis, erythropoietin to treat ischemia, nerve growth factor to treat Alzheimer's disease, and beta-endorphin to alleviate pain.

In treating diseases, the strategy of cell transplantation is critical for the success of cell-based drug delivery as the ability of cells to secrete the desired drug molecules. Cells directly injected into the body experience less than desired therapeutic efficacy for many reasons, including immune rejection and rapid decrease in cell viability. Immunosuppression drugs may reduce immune rejection of transplanted cells, but such drugs have serious side effects. Furthermore, transplanted cells not surrounded by the proper environment rapidly lose viability and therefore lose their ability to produce and secrete drug molecules.

Encapsulating cells into various biomaterials prior to transplantation can reduce immune response while simultaneously allowing creation of a cell-supporting microenvironment. Synthetic hydrogels have properties resembling naturally occurring extracellular matrices. Viability of transplanted cells using synthetic hydrogels varies depending upon properties of the gel such as porosity, isotonicity, immunogenicity, mechanical stiffness, degradation rate, and diffusion rate.

Before any hydrogel can be utilized for delivering viable cells, a method of synthesizing the gel and encapsulating the cells must be developed that causes minimal harm to the cells. Most known methods of polymerizing hydrogels involves chemical reactions, temperatures, and other energies that are destructive to biological organisms, even if the polymerized end product is relatively inert. Therefore, optimizing encapsulation while maximizing cell viability represents an ongoing goal of this technology.

Two categories of encapsulation have been identified: Macro encapsulation involves polymerization of hydrogels followed by surrounding large numbers of cells with the pre-polymerized hydrogel, usually by injecting the cells into the gel. With macro encapsulation migration of the cells into the hydrogel structure is minimal or non-existent. Microencapsulation, on the other hand, results in individual cells or relatively small numbers of cells interspersed within the polymerized hydrogel matrix. All known methods of microencapsulation require inclusion of the cells in a pre-polymerized emulsion during the polymerization reaction used to create the hydrogel.

Due to the biologically destructive environment created by polymerization reactions, methods of microencapsulation of viable whole cells after polymerization would be advantageous.

### SUMMARY OF THE INVENTION

An object of the present invention provides methods for using biodegradable hydrogels for concurrent delivery of multiple therapeutic agents. More specifically, the present invention discloses new methods for encapsulation of multiple therapeutic agents within hydrogels.

These aspects, and others, that will become apparent to the artisan upon review of the following description, can be accomplished through the following process: The process disclosed involves formulating a starch-derived polymeric network, and diffusing more than one therapeutic agent into the network. Alternatively, the process involves formulating a starch-derived polymeric network that incorporates one or more therapeutic agents into the network, followed by diffusing one or more therapeutic agents into the network.

Certain preferred embodiments of the process result in a hydrolysable starch-derived polymeric substance combined with multiple therapeutic agents that are particularly well suited for sustained delivery of said therapeutic agents.

Another object of the present invention provides methods for using biodegradable hydrogels for delivery of whole living cells as alternative drug delivery devices. More specifically, the present invention discloses new methods for encapsulation of whole living cells within hydrogels after said hydrogels have been polymerized, thereby increasing the viability of cells to be transplanted, allowing more sensitive types of cells to be successfully encapsulated, and increasing the types of hydrogels that can be used for cell transplantation.

These aspects, and others, that will become apparent to the artisan upon review of the following description, can be accomplished through the following process: The process disclosed involves polymerizing a hydrogel using less than a total volume of water required to create an equilibrium state between the polymerized gel and water, then submerging the polymerized hydrogel in cell culture medium containing cells to be transplanted and incubating the submerged gel in the medium. Cells and culture media diffuse into the gel as the gel swells. Cells are then transplanted into target tissue by injection or other means.

It is to be understood that both the foregoing general description and the following detailed description provide embodiments of the invention and are intended to provide an overview or framework of understanding the nature and character of the invention as it is claimed.

### DESCRIPTION OF THE DRAWING

FIG. 1 is a block diagram representing an overview of the processes involved in encapsulation of whole viable cells within a hydrogel matrix.

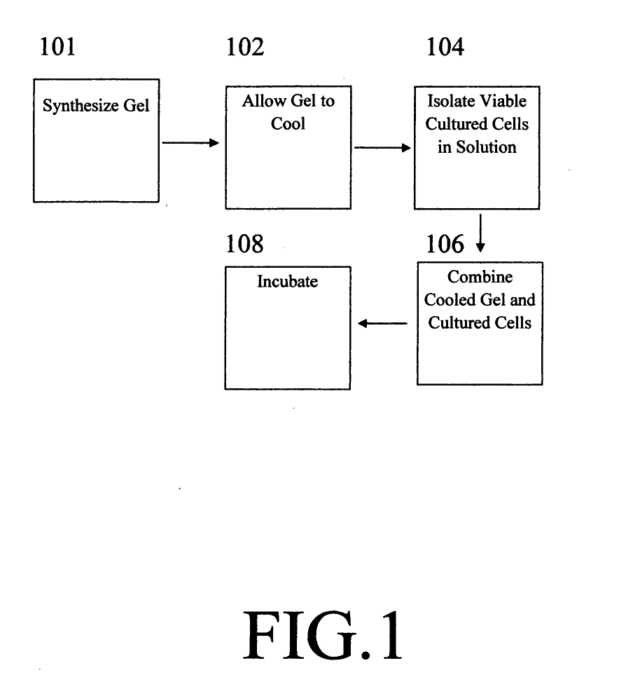

### DETAILED DESCRIPTION OF THE INVENTION

The present invention provides methods for formulating multiple therapeutic agents into a hydrogel for biomedical applications, including but not limited to delivery systems for whole viable biological cells, which is described more fully hereinafter. This invention may be embodied in many different forms and should not be construed as limited to the specific embodiments described herein.

Hydrogels are crosslinked polymeric structures formed by either covalent bonds produced by the simple reaction of one or more comonomers, physical crosslinks from entanglements, association bonds such as hydrogen bonds or strong van der Waals interactions between chains, or crystallites bringing together two or more macromolecular chains that are able to swell in water. Hydrogels can be categorized by their method of preparation, ionic charge, or physical structure features. Hydrogels can be homopolymer, copolymer, multipolymer, or interpenetrating polymeric networks depending on the method of preparation. Homopolymer hydrogels are crosslinked networks of one type of hydrophilic monomer unit. Copolymer hydrogels are produced by crosslinking two comonomer units. Multipolymer hydrogels are produced from three or more comonomers that have been reacted together to form a network. Interpenetrating networks are usually formed by producing the first network and then swelling that network in the other monomer. The monomer then reacts to form a second network structure that is intertwined with the first network. Because of monomer functionalities and chemical nature of the functional groups within the chains charge effects occur producing neutral, anionic, cationic, or ampholytic gels. Crosslinked networks have amorphous, semi-crystalline, or complexed physical structures. In amorphous hydrogels, the macromolecular chains are arranged randomly. Semicrystalline hydrogels have dense regions of ordered macromolecular chains, or crystallites, that are arranged randomly. Complexed structures are those formed by hydrogen bonds or chemical complexes of one monomer with another or of the chains of the network.

The terms crosslink and junction or tie-point indicate the connection points of the polymer chains that make up the network. In one embodiment Hydrogel forming crosslinks are carbon atoms. In another embodiment Hydrogel forming crosslinks are small chemical bridges with molecular weights much smaller than those of the polymer chains. In yet another embodiment Hydrogel forming crosslinks are an association of macromolecular chains caused by van der Waals forces or an aggregate formed by hydrogen bonds. In another embodiment Hydrogel forming crosslinks are a combination of carbon atoms, small chemical bridges, and van der Waals aggregates.

Methods of preparation of the initial networks include chemical crosslinking, photopolymerization, or irradiative crosslinking. Hydrogels are produced by swelling crosslinked structures in water or biological fluids. Because hydrogels must be brought in contact with water during the preparation to yield the final swollen network structure, their physical behavior is largely dependent on their equilibrium and dynamic swelling behavior. Usually, a hydrophilic crosslinked network is placed in water, and because of the thermodynamic compatibility of the macromolecular chains interacting with the solvent molecules the network then expands to its swollen state.

Hydrogels in pharmaceutical applications have become popular in recent years because of their biocompatibility. Pharmaceutical hydrogel systems include matrices that have a drug or whole cell incorporated into them. A system prepared by incorporating a cell into a hydrophilic polymer is swollen when brought in contact with water, cell culture media, or biological fluids. The swelling process proceeds toward equilibrium at a rate dependent on the water activity in the system and the structure of the polymer. If the polymer is crosslinked or if it is of sufficiently high molecular weight so that chain entanglements can maintain structural integrity, the equilibrium state is an aqueous swollen gel. The equilibrium water content of such hydrogels can vary from 30% to 98%. If the dry hydrogel contains a water soluble drug, the drug is essentially immobile in the matrix, but begins to diffuse out as the polymer swells. Drug release then depends on diffusion outward through the swollen gel.

In an exemplary embodiment of the present invention the crosslinked starch gels are synthesized by first dissolving glucaric acid potassium salt in deionized water. Amylopectin dissolves only in cold water solutions while amylose dissolves only in hot water solutions, so each glucaric acid solution is allowed to cool to room temperature before adding the amylopectin powder and then the solution is heated before adding the amylose. All solutions are constantly stirred to prevent sedimentation. The amylose aggregates instantly if the water temperature is too low limiting the diffusion of water into the system, but the amylopectin gels when the solution reaches approximately 75 degrees C. The amylopectin glucaric acid solution is heated to approximately 55 degrees C. before the amylose powder is incorporated into the solution. The crosslinked starch solution is then heated until the viscosity of the system increases indicating the formation of the gel. The gel is then removed from the heat source and allowed to cool to room temperature. The resulting synthesized gel are white, opaque, highly viscous, rubber-like gels. The following reactions occur during this synthesis:

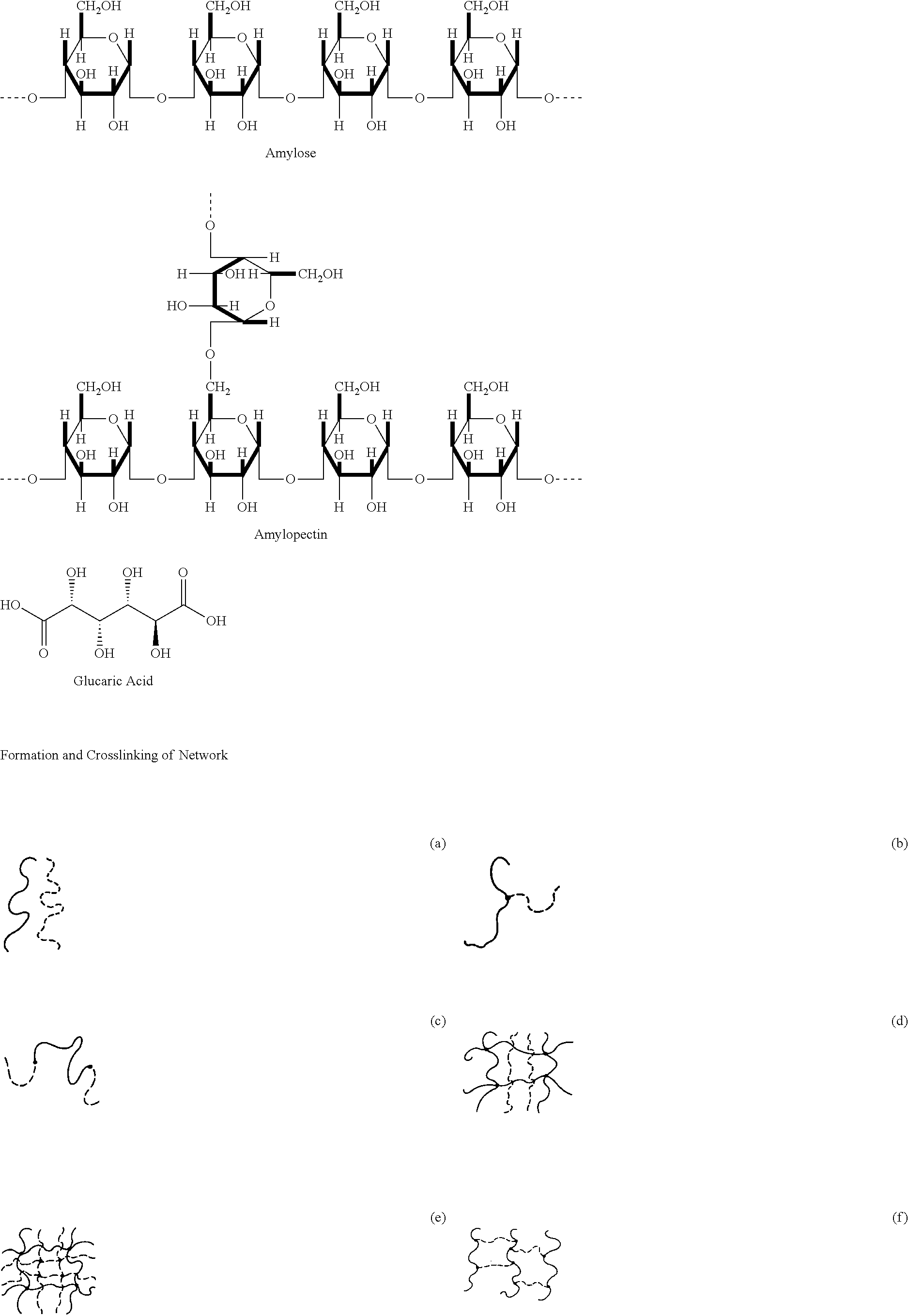

Concentrations of crosslinker and starch, as well as water volumes are varied to control the mechanical and degradation properties, as well as swell volume and rate. For example, lower weight percent of polymer, for example 3-10%, have a much higher swell ratio than gels with higher percentages of polymer. Higher polymer concentrations increase degradation times.

Because of the physical properties of such hydrogels, they can be locally administered at the site of a tumor to be treated. The physical properties, including dispersion rate, of such hydrogels can be manipulated in several ways. (See Barker, E D “A novel biodegradable hydrogel for biomedical applications including the treatment of malignant tumors and prevention of metastatic disease,” Masters Thesis. University of Tennessee at Knoxville, August 2007). For example, the cross-link can change the pore size of the matrix thereby impeding diffusion of the therapeutic agents out of the gel matrix.

In various embodiments the process results in a hydrogel that includes at least four different chemical substances: (1) a starch-derived polymeric network, (2) a cross-link, (3) a first therapeutic agent, and (4) a second therapeutic agent. The starch-derived polymetric network is cross-linked to facilitate the controlled release in vivo of the first and second therapeutic agents. In at least some of these embodiments, the starch-derived polymeric network is cross-linked also to facilitate the retention in vitro of the first and second therapeutic agents. The therapeutic agents complex with the starch-derived hydrogel by one of three mechanisms: the therapeutic agents can be incorporated into the matrix of the gel network; the therapeutic agents can complex within the molecular structure of the individual polymer chains comprising the network; or the therapeutic agents can react with the main chain backbone of the polymers comprising the network.

In one embodiment the therapeutic agents are loaded into the hydrogel during the synthesis of the network. In another embodiment the therapeutic agents are loaded into the hydrogel after the network has formed. In yet another embodiment the therapeutic agents are loaded into the hydrogel using a combination of the above alternatives, depending upon the type of agent being loaded. For example, a protein-like therapeutic agent or a viable whole cell would not be able to withstand the heat required to form the hydrogel network, so it is added after the gel has been formed.

In one embodiment the gel is synthesized before incorporation of the therapeutic agents and then submerged in solution containing the compounds to be released. In another embodiment the compounds are added to the pre-polymer solution and the gel synthesized around the active pharmaceutical ingredients.

The polymer concentration of the hydrogel are varied to control the release kinetics of the compounds from the system. The Amygel system is designed to accommodate multiple therapeutic compounds and to control the release of those compounds from one of at least three sites within the polymer matrix. The polymeric chains within the system form a 3 dimensional network that swells in solvent and encapsulate therapeutic compounds. These compounds are then release from the system via simple diffusion. The shape of the polymer chains, especially the helices of the Amylopectin molecules, allows for complexation of drug compounds within the polymer chains that make up the network of the gel system. Also, there are reactive functional groups present on the mainchain backbone of the polysaccharides as well as the reactive ends of the crosslinker, specifically the alcohol groups of the starch and the carboxyl groups of the di-carboxyllic acid, that allow for covalent bonding of certain drug compounds to the polymer network. Each of these interactions allows for better controlled release kinetics of compounds from the drug delivery system, and the material are engineered with any combination of these interactions to produce the desired release effects appropriate for specific drug delivery applications.

To load the gel after synthesis, a simple diffusion method is used to dissolve the therapeutic agent in PBS and add the gel to the solution. The active agent then diffuses into the gel and becomes complexed within the gel matrix.

The cross-link properties change the mechanical properties of the starch-derived polymeric network, thereby enhancing the ability of the network to retain therapeutic agents. Such changes affect the helical regions of the starch-derived polymeric network, thereby enhancing the ability of the network to retain the therapeutic agents. It should be noted that whether or not the therapeutic agents are complexed within the structure of the starch-derived polymeric network determines whether diffusion or degradation rate of the network is the primary factor in determining release rate of the therapeutic agents. It should also be noted that hydrogels are created that include a first therapeutic agent that is incorporated into the structure of the starch-derived polymeric network while a second therapeutic agent is not incorporated into the structure of the starch-derived polymeric network. Such a confirmation results in considerable differentials in release rates. Such different release rates provide advantageous options for particular therapy regimens.

In another preferred embodiment, once the hydrogel has been polymerized and cross linked [101] it is allowed to cool to 37 degrees C. [102]. Viable cells to be microencapsulated in the gel are trypsonized maintained in appropriate aqueous cell culture media [104]. The gel and the cells are then combined [106] by submerging the gel in the aqueous cell culture media containing cells to be transplanted. The hydrogel and media containing cells are then incubated [108] at 37 degrees Celsius overnight. Diffusion of the aqueous cell culture media causes swelling of the polymerized hydrogel which increases the hydrogel's porosity and allows cells to diffuse into gel's matrix along with the cell culture media.

In yet another alternative embodiment of the present invention hydrogel is polymerized and submerged in water until maximum swell equilibrium is achieved. 100 microliters of cells in aqueous cell culture solution at a concentration of 50,000 cells per microliter are then injected into the gel. The macroencapsulated cells and hydrogel is then injected into the target tissue, which can be subcutaneous, intraperitoneal, or intra muscular.

### SPECIFIC EXAMPLES

In order that the invention described herein may be more fully understood, the following examples are set forth. It should be understood that these examples are for illustrative purposes only and are not to be construed as limiting the scope of this invention in any manner.

### Example I

Microencapsulation of Neurons into Amygel:

The following is an exemplary description of the use of Amygel as a polymeric scaffold for the regeneration of neurons caused by degenerative disease states.

Cell Harvesting and Culture: Brains are removed from 1-3 day old Sprawgue-Dawley rats. The cerebral cortices are dissected out in 80% Ca/Mg-free Hank's balanced salt solution containing 4 mM NaHCO3, 5 mM HEPES buffer, and 20% fetal bovine serum. The meninges are removed and the cortex is cut into sections. The cortex is washed 3 times with Hank's balanced salt solution and incubated for 5 min at 37 C in digestion media. Digestion Media: 5 mg/mL trypsin type XI, 0.5 mg/mL DNase type IV, 137 mM NaCl, 5 mM KCl, 7 mM Na2HPO4, and 25 mM HEPES@pH 7.2. After digestion, the separate cells are harvested by centrifugation, 1000 rpm at 4 C for 10 min. Gel Synthesis:

A 3% prepolymer solution of 2% by weight amylopectin and 1% soluble starch, and 1% glucaric acid potassium salt in 3 mL of phosphate buffered saline is mixed. The solution is vigorously stirred. The gel is synthesized by microwaving for 10 seconds on high power in a petri dish creating a layer of gel on the bottom of the dish. The gel is allowed to cool before seeding the cells.

Cell Seeding into the Gel: Approximately 10 mL plating media is added to the gel dishes. Plating Media: 90% 28 nM Glucose, 2.5 mM NaHCO3, 1 mg/10 mL of transferring, 20 nM glutamine, 0.73 mM HCl, and 2.5 mg/100 mL in Minimum Essential Medium with Earle's salts, without L-glutamine or phenol red and 10% fetal bovine serum. After the addition of the plating medium, the cells that were centrifuged are added. The cells are allowed to incubate in the gel plates for 24 hours before removing the plating media and adding the feeding media. Feeding media: 28 mM Glucose, 2.5 mM NaHCO3, 1 mg/10 mL transferring, 30 mM glutamine, 1 mL/100 mL B27 50× supplement, and 0.84 mM cytosine arabinoside in Minimum Essential Medium. Feeding culture media is replaced weekly until implantation.

Gel Implantation: the cell loaded gels are implanted into the forebrain of healthy Sprawgue-Dawley rats. 150 g male rats are used. Sterile technique and a surgical microscope are used to implant the gels. Isofluorane anesthesia is used, 3% initially and then 1% for the duration of the procedure. An incision is made in the scalp and then using a small bore size dental drill a hole 2 mm is made posterior to the bregma and 4 mm from the midline. 8 uL of cell loaded gel is inject using a 27 gauge needle and Hamilton syringe. The scalp is closed with an Ethilon suture. Buprenex (0.1 mg/kg, im) is used for analgesia twice daily for two days post implantation.

### Example II

Microencapsulation of Embryonic Stem Cells and subsequent delivery of dopamine via the Amygel drug delivery system:

Cell Culture: Mouse ES cell aggregates that are prepared by the hanging-drop culture method are cultured in conditioned medium of PA6 cells to differentiate the ES cells into dopaminergic neurons. Alternatively, cell line EB5, a mouse embryonic stem cell line derived from the E14tg2a ES cell line available from RIKEN is used. The cells that carry the blasticidin S-resistant selection marker gene driven by the Oct3/4 promotor are maintained on gelatin-coated dishes in Glasgow minimum essential medium, GMEM containing 20 ug/mL blasticidin S (Invitrogen) to eliminate differentiated cells. Other supplements for the media include 1% fetal bovine serum, 10% knockout serum replacement, 0.1 mM nonessential amino acid all from Invitrogen and 1 mM private, 0.1 mM 2-mercaptoethanol, and 2000 U/mL ESGRO. PA6 cells, skull bone marrow cells, are grown on culture dishes in minimum essential medium alpha supplemented with 10% FBS, 50 U/mL penicillin, and 50 ug/mL streptomycin. Confluent dishes are rinsed 3 times in phosphate-buffered saline with calcium and magnesium ions. The cells are maintained in GMEM supplemented with 5% KSR, 0.1 mL nonessential amino acid, 1 mM pyruvate, 0.1 mM 2-mercaptoethanol, and 10 ug/mL heparin for 2 days. The supernatant collected from this protocol is used as the conditioned medium. The conditioned medium is filtered through a 0.22 micron filter before adding to the ES cultures. To form the cell aggregates, the ES cells are trypsinized and collected using 0.25% trypsin and EDTA solution. Cells are seeded at 25 cells per micro liter into a hanging drop of the 20 micro liters of conditioned medium. The aggregates are allowed to differentiate for 2 days. The aggregates are collected and cultured in dishes in the same conditioned medium for 14 days under free-floating conditions in air containing 5% CO2 at 37 C. The culture medium is changed every 2-3 days.

Gel Synthesis: A 3% prepolymer solution of 2% by weight amylopectin and 1% soluble starch, and 1% glucaric acid potassium salt in 3 mL of phosphate buffered saline is mixed. The solution is vigorously stirred. The gel is synthesized by microwaving for 10 seconds on high power in a glass vial, or in a water bath (at ˜76 C) with constant stirring to prevent sedimentation. The gel is allowed to cool before seeding the cells.

Cell Seeding into the gel: After the synthesized gel has cooled to 37 C, the cell aggregates that have been cultured for 16 days are added and mixed with the synthesized gel. The gel/cell solution is replated in petri dishes containing the conditioned medium with 50 U/mL and 50 ug/mL streptomycin until ready to implant. The polymer delivery system is cultured for 6 weeks before implanting. Approximately 8 uL of the gel with microencapsulated cells is implanted intracranially by injection.

### Example III

Amygel as a polymeric scaffold for the treatment of spinal cord injuries: This example describes a method resulting in macroencapsulation of viable cells.

Cell Culture: The Bone Marrow Stromal Cell model proposed by Bakshi (Journal of Neurosurgery 2004) in which both host and transplanted cells belonged to the same inbred strain of Fisher-344 rats is used, thereby eliminating the necessity of immunosuppression. Cells from inbred, adult transgenic Fisher rat bone marrow obtained in the femur and tibia are isolated. The bones are resected and rinsed in saline. The epiphysial plates are then excised and the bone marrow is flushed out using a syringe and needle with Hank buffered saline solution. The cells are pooled, counted, and centrifuged at 600 G for 10 minutes. The cells are then plated at 120×1⁰⁶ cells/cm2 in 10% fetal bovine serum, 45% Hams F-12, 45% a-MEM, and supplemented with 100 U/ml penicillin and 100 mg/ml streptomycin sulfate. Cells are then incubated in a humidified atmosphere with 5% CO2 at 37° C. After 8 days, nonadherent cells are removed. The remaining cells are then detached with 0.05% trypsin/0.53 mM ethylenediaminetetraacetic acid, and replated at 2000 cells/cm2. The cultures are passed at 4-day intervals. The resulting stromal cell fraction represents a population of multipotential stemlike cells that have been well characterized in their ability to proliferate and differentiate into various phenotypes. Cells that have been passaged fewer than five times are used to maintain their phenotypic and growth potential. In order to differentiate the cells they are co-cultured and implanted with lineage restricted neural precursor cells that will express neurotrophic factors required for the sustained viability of the stromal cells in vivo. Embryos from transgenic Fischer-344 rats expressing the alkaline phosphatase marker gene removed on embryonic Day 13.5 are used to create lineage restricted cells. The embryos are transferred to dishes containing, Hank buffered saline solution. Caudal regions of the embryonic spinal cords are dissected and incubated in 0.05% trypsin/ethylenediaminetetraacetic acid for 7 minutes, and then surrounding connective tissues are removed. The cords are placed in fresh medium, centrifuged at 1000 rpm for 5 minutes, and resuspended in fresh culture medium. The media recipe: Dulbecco modified Eagle medium/F-12 supplemented with bovine serum albumin, B27, 500 IU/ml penicillin, 500 mg/ml streptomycin, N2 supplement, basic fibroblast growth factor (10 ng/ml), and neurotrophic factor-3 (20 ng/ml). Gently triturate cords to a single cell suspension, and plate on poly-L-lysine and laminin-coated flasks. Cells are cultured for 48 hours at 37° C. and 5% CO2 prior to transplantation.

Spinal Cord Injury Model: 344 Fischer rats are anesthetized using an intraperitoneal injection of xylazine-acepromazine-ketamine. A midline vertical incision is made over the cervical spine and the paraspinal muscles are retracted laterally. A C3-4 laminectomy is performed using an operating microscope. The dura mater is opened longitudinally, and the right lateral funiculus of the spinal cord is excised using microsurgical technique at C-3. The wound is closed with 9-0 nylon.

Gel Synthesis: A 3% prepolymer solution of 2% by weight amylopectin with 1% soluble starch, and 1% glucaric acid potassium salt in 3 mL of phosphate buffered saline is mixed. The solution is stirred vigorously. The gel is synthesized by microwaving for 10 seconds on high power in a glass vial, or in a water bath (at approximately 76 degrees C.) with constant stirring to prevent sedimentation. The gel is allowed to cool to 37 degrees C. before seeding the cells.

Cell Loading: One million lineage restricted cells and one million bone marrow stromal cells are resuspended in 20 ul of complete medium, and injected into 30 uL of gel. The 50 uL total volume is then implanted into the cavity at the site of the injury.

Gel Implantation: The cell seeded gel is implanted 24 hours after the spinal cord injury procedure using a puncture technique. Under isofluorane anesthesia and using a stereotactic frame, the surgical wound is re-opened by making a longitudinal incision over the C3-4 spinous processes and the skin is retracted. A 25-gauge needle is used to inject the gel at the intradural space. Proper needle placement is confirmed and the CSF present in the needle hub is aspirated using a micropipette. The 50 uL of loaded hydrogel is then injected. The wound is closed as before with 9-0 nylon.

### Example IV

The following is an exemplary description of the formulation of Amygel and two therapeutic agents:

Two drugs are incorporated into the Amygel system: Doxorubicin Hydrochloride, a broad spectrum anti-neoplastic agent, and Metformin, a compound thought to have unique properties against cancer stem cells. Both drugs are solubilized in aqueous or a combination of organic and aqueous solvent before being incorporated into the gel system. The anti-neoplastic agents are dissolved in solvent. The Amylopectin, Soluble Starch, and Glucaric Acid Potassium Salt powders are added to the solution to obtain a final polymer concentration of 3-25% weight/volume depending on the preferred release rate. The pre-polymer solution is loaded into a sterile syringe. The syringe is wrapped to protect the Doxorubicin from photo degradation and the polymer is synthesized via microwaving.

### Example V

Another exemplary method provides for the formulation of Amygel and three therapeutic agents:

Methotrexate, Doxorubicin, and Mitoxantrone are three commonly used anti-neoplastic agents that have been investigated for simultaneous delivery. The drugs are dissolved in Dimethyl Sulfoxide. In a separate container the gel is synthesized by microwave or conventional heating at concentrations to produce the desired release effects. The swollen gel is then submerged in the drug/DMSO solution and incubated to allow the active pharmaceutical ingredients to diffuse into the gel structure thereby incorporating multiple compounds to be released from the system.

It is apparent that many modifications and variations of this invention as set forth above may be made without departing from the spirit and scope. The specific embodiments described are given by way of example only, and the invention is limited only by the terms of the appended claims.

What is claimed is:

1. A process for concurrently delivering multiple therapeutic agents for biomedical applications comprising the steps of:
a) polymerizing a hydrogel;
b) combining the hydrogel with more than one therapeutic agent; and
c) delivering the combined hydrogel with more than one therapeutic agent to tissue within a living organism.

2. The process of claim 1 further comprising the step a2) of dissolving more than one therapeutic agent in a solubilizing agent.

3. The process of claim 2 wherein the solubilizing agent is dimethyl sulfoxide.

4. The process of claim 1 wherein one of the more than one therapeutic agents is a biological cell.

5. The process of claim 1 wherein more than one of the more than one therapeutic agents is a biological cell.

6. The process of claim 1 wherein the more than one therapeutic agents comprises at least one stem cell.

7. The process of claim 1 wherein the more than one therapeutic agents comprises at least one neuron.

8. The process of claim 1 wherein the living organism is a human.

9. A process for concurrently delivering multiple therapeutic agents for biomedical applications comprising the steps of:
a) combining an unpolymerized hydrogel solution with more than one therapeutic agents;
b) polymerizing the hydrogel; and
c) delivering the hydrogel with more than one therapeutic agent to cells within a living organism.

10. The process of claim 9 wherein the unpolymerized hydrogel solution comprises an aqueous solvent, an amylase, an amylopectin, a soluble starch, and a glucaric acid salt.

11. The process of claim 9 wherein the hydrogel comprises a starch-derived polymeric network and at least one cross-link.

12. The process of claim 9 wherein at least one of the more than one therapeutic agents is an anti-neoplastic agent.

13. A process of microencapsulating biological cells for biomedical applications comprising the steps of:
a) growing a population of biological cells within a liquid nutrient media;
b) polymerizing a hydrogel;
c) combining the hydrogel the population of biological cells and the nutrient media; and
d) incubating the hydrogel the population of biological cells and the nutrient media.

14. The process of claim 13 further comprising the step of delivering the combined hydrogel, population of biological cells, and the nutrient media to a tissue within a living organism.

15. The process of claim 13 wherein the population of biological cells comprises stem cells.

16. The process of claim 13 wherein the population of biological cells comprises neurons.
